(19) United States
(12) Patent Application Publication
(10) Pub. No.: US 2023/0225403 A1
(43) Pub. Date: Jul. 20, 2023

(54) AEROSOL DELIVERY DEVICE

(71) Applicant: NICOVENTURES TRADING LIMITED, London (GB)

(72) Inventors: Ping-Chou Chen, Hampshire (GB); Steven Michael Schennum, Plainfield, IL (US); Matthew Joel Nettenstrom, Bartlett, IL (US)

(21) Appl. No.: 17/928,097

(22) PCT Filed: May 27, 2021
(86) PCT No.: PCT/IB2021/054665

§ 371 Date: Nov. 28, 2022

Related U.S. Application Data
(60) Provisional application No. 63/032,153, filed on May 29, 2020.

(51) Int. Cl. A24F 40/42; A24F 40/485; A61M 11/04

(52) U.S. Cl. CPC A24F 40/42; A24F 40/485; A61M 11/042

(57) ABSTRACT

The present disclosure provides an aerosol delivery device and a cartridge for an aerosol delivery device. The cartridge can comprise a body defining a reservoir and defining an aerosol pathway in fluid communication with an aerosol outlet. The cartridge can further include one or more of an air entry, a splitter configured to split air entering the air entry into a plurality of air passages, an atomizer, and an aerosol forming chamber where air from the plurality of air passages is configured to mix with vapor formed by the heater to form an aerosol. Preferably, the aerosol forming chamber can be in fluid connection with the aerosol pathway. The present disclosure further can include a control device configured to receive, power, and control aerosol formation by the cartridge.

### TECHNOLOGY FIELD

The present disclosure relates to aerosol delivery devices such as smoking articles, and more particularly to aerosol delivery devices that may utilize electrically generated heat for the production of aerosol (e.g., smoking articles commonly referred to as electronic cigarettes). The smoking articles may be configured to heat an aerosol precursor, which may incorporate materials that may be made or derived from tobacco or otherwise incorporate tobacco, the precursor being capable of forming an inhalable substance for human consumption.

### BACKGROUND

Many smoking devices have been proposed through the years as improvements upon, or alternatives to, smoking products that require combusting tobacco for use. Many of those devices purportedly have been designed to provide the sensations associated with cigarette, cigar, or pipe smoking, but without delivering considerable quantities of incomplete combustion and pyrolysis products that result from the burning of tobacco. To this end, there have been proposed numerous smoking products, flavor generators, and medicinal inhalers that utilize electrical energy to vaporize or heat a volatile material, or attempt to provide the sensations of cigarette, cigar, or pipe smoking without burning tobacco to a significant degree. See, for example, the various alternative smoking articles, aerosol delivery devices, and heat generating sources set forth in the background art described in U.S. Pat. No. 7,726,320 to Robinson et al., U.S. Pat. App. Pub. No. 2013/0255702 to Griffith Jr. et al., and U.S. Pat. App. Pub. No. 2014/0096781 to Sears et al., which are incorporated herein by reference in their entireties. See also, for example, the various types of smoking articles, aerosol delivery devices, and electrically powered heat generating sources referenced by brand name and commercial source in U.S. patent application Ser. No. 14/170,838 to Bless et al., filed Feb. 3, 2014, which is incorporated herein by reference in its entirety. It would be desirable to provide an aerosol delivery device with advantageous usability features.

### BRIEF SUMMARY

The present disclosure relates to aerosol delivery devices and elements of such devices. The disclosure particularly relates to an aerosol delivery device and a cartridge for use in an aerosol delivery device. In this regard, various embodiments of the disclosure provide an aerosol delivery device and/or a cartridge suitable for use with such an aerosol delivery device.

In one or more embodiments, the present disclosure can provide a cartridge for use in an aerosol delivery device, the cartridge comprising: a body defining a reservoir and defining an aerosol pathway in fluid communication with an aerosol outlet; an air entry; an atomizer; an aerosol forming chamber; and an air pathway extending from the air entry, through the aerosol forming chamber, the aerosol pathway, and the aerosol outlet; wherein the cartridge includes one or more splitting elements effective to split an air stream at least once while passing through the air pathway. In additional embodiments, the cartridge can be further defined in relation to one or more of the following statements, which statements can be defined in any number and/or order.

The one or more splitting elements can be positioned so that the air stream is split prior to entering the aerosol forming chamber.

The one or more splitting elements can be positioned so that the air stream is split into a plurality of air streams that are directed toward a surface of the atomizer at a plurality of points that are laterally positioned relative to a geometric center of the surface of the atomizer.

The atomizer can include a surface with a midline that extends from a front edge of the atomizer to a back edge of the atomizer and that is approximately centrally located between a first end of the atomizer and a second end of the atomizer, and wherein the one or more splitting elements is positioned so that the air stream is split into two air streams that are directed toward the surface of the atomizer on each of two sides of the midline.

The one or more splitting elements can comprise a wedge that is substantially centrally located above air entry.

The cartridge can comprise an upper frame member positioned proximate the reservoir, a lower frame member engaging an opening at an end of the body opposite from the aerosol outlet, and a bottom seal positioned in the body between the upper frame member and the lower frame member.

The air entry can be defined in the lower frame member.

The bottom seal can include two air apertures.

The one or more splitting elements can comprise a central member positioned in the bottom seal between the two air apertures.

The two air apertures can be substantially symmetrically arranged relative to the air entry.

The aerosol forming chamber can be defined between the upper frame member and the bottom seal.

The bottom seal can comprise an island positioned between the aerosol forming chamber and the aerosol pathway of the body.

The island can be effective to split an aerosol stream from the aerosol forming chamber into two streams prior to passage of the aerosol stream into the aerosol pathway.

The bottom seal can comprise a bottom wall that defines a floor across at least a portion of a top surface of the bottom seal.

The floor of the bottom seal can comprise one or more components effective to trap liquid therein.

The one or more components effective to trap liquid can comprise one or more baffles that individually completely surround one or more openings though the bottom seal.

The cartridge can comprise baffles that individually completely surround a plurality of air apertures through the bottom seal.

The bottom seal can comprise an outer edge that can be in contact with an inner surface of an outer wall of the tank body.

The outer edge of the bottom seal can comprise a plurality of ribs.

The cartridge can include at least two separate splitting elements effective to split the air stream at least twice while passing through the air pathway.

The at least two separate splitting elements can be positioned in the cartridge so that the air stream is split in a direction that is substantially parallel to a longitudinal axis of the cartridge and is split in a direction that is substantially orthogonal to the longitudinal axis of the cartridge.

The atomizer can comprise a heater.

The atomizer further can comprise a liquid transport element.

In further embodiments, the present disclosure can provide a cartridge for use in an aerosol delivery device. In particulate, the cartridge can comprise: a body defining a reservoir and defining an aerosol pathway in fluid communication with an aerosol outlet; an air entry; a splitter configured to split air entering the air entry into a plurality of air passages; a heater; and an aerosol forming chamber where air from the plurality of air passages is configured to mix with vapor formed by the heater to form an aerosol, the aerosol forming chamber being in fluid connection with the aerosol pathway.

In still further embodiments, a cartridge for use in an aerosol delivery device can comprise: a body including an outer wall and defining a reservoir, an aerosol pathway in fluid communication with an aerosol outlet, and an opening at a bottom end thereof, the opening being defined by the outer wall; and a sealing member positioned within the body between the reservoir and the opening at the bottom of the body, the sealing member having an outer edge that is in contact with an inner surface of the outer wall of the body, wherein the sealing member includes a bottom wall with at least one aperture therethrough, the at least one aperture being surrounded by a baffle that extends upward from the bottom wall.

In one or more embodiments, the present disclosure further can provide methods for forming a cartridge for an aerosol delivery device. For example, such method can comprise: performing a first injection molding step with a first polymeric material, the first injection molding step being effective to form a body defining at least a reservoir and an aerosol pathway separated from the reservoir; and performing a second injection molding step with a second polymeric material. that differs from the first polymeric material in at least one aspect, the second injection molding step being effective to cover at least a portion of the body with a layer of the second polymeric material. For example, the first polymeric material can be transparent or translucent, and the second polymeric material can be opaque or colored. Further, the method can include adding a framing component to a mouthend of the body after performing the first injection molding step and prior to performing the second injection molding step.

The present disclosure includes, without limitation, the following embodiments.

Embodiment 1: A cartridge for use in an aerosol delivery device, the cartridge comprising: a body defining a reservoir and defining an aerosol pathway in fluid communication with an aerosol outlet; an air entry; an atomizer; an aerosol forming chamber; and an air pathway extending from the air entry, through the aerosol forming chamber, the aerosol pathway, and the aerosol outlet; wherein the cartridge includes one or more splitting elements effective to split an air stream at least once while passing through the air pathway.

Embodiment 2: The cartridge of Embodiment 1, wherein the one or more splitting elements is positioned so that the air stream is split prior to entering the aerosol forming chamber.

Embodiment 3: The cartridge of any of Embodiments 1 and 2, wherein the one or more splitting elements is positioned so that the air stream is split into a plurality of air streams that are directed toward a surface of the atomizer at a plurality of points that are laterally positioned relative to a geometric center of the surface of the atomizer.

Embodiment 4: The cartridge of any of embodiments 1 to 3, wherein the atomizer includes a surface with a midline that extends from a front edge of the atomizer to a back edge of the atomizer and that is approximately centrally located between a first end of the atomizer and a second end of the atomizer, and wherein the one or more splitting elements is positioned so that the air stream is split into two air streams that are directed toward the surface of the atomizer on each of two sides of the midline.

Embodiment 5: The cartridge of any of embodiments 1 to 4, wherein the one or more splitting elements comprises a wedge that is substantially centrally located above air entry.

Embodiment 6: The cartridge of any of embodiments 1 to 5, wherein the cartridge comprises an upper frame member positioned proximate the reservoir, a lower frame member engaging an opening at an end of the body opposite from the aerosol outlet, and a bottom seal positioned in the body between the upper frame member and the lower frame member.

Embodiment 7: The cartridge of any of embodiments 1 to 6, wherein the air entry is defined in the lower frame member.

Embodiment 8: The cartridge of any of embodiments 1 to 7, wherein the bottom seal includes two air apertures.

Embodiment 9: The cartridge of any of embodiments 1 to 8, wherein the one or more splitting elements comprises a central member positioned in the bottom seal between the two air apertures.

Embodiment 10: The cartridge of any of embodiments 1 to 9, wherein the two air apertures are substantially symmetrically arranged relative to the air entry.

Embodiment 11: The cartridge of any of embodiments 1 to 10, wherein the aerosol forming chamber is defined between the upper frame member and the bottom seal.

Embodiment 12: The cartridge of any of embodiments 1 to 11, wherein the bottom seal comprises an island positioned between the aerosol forming chamber and the aerosol pathway of the body.

Embodiment 13: The cartridge of any of embodiments 1 to 12, wherein the island is effective to split an aerosol stream from the aerosol forming chamber into two streams prior to passage of the aerosol stream into the aerosol pathway.

Embodiment 14: The cartridge of any of embodiments 1 to 13, wherein the bottom seal comprises a bottom wall that defines a floor across at least a portion of a top surface of the bottom seal.

Embodiment 15: The cartridge of any of embodiments 1 to 14, wherein the floor of the bottom seal comprises one or more components effective to trap liquid therein.

Embodiment 16: The cartridge of any of embodiments 1 to 15, wherein the one or more components effective to trap liquid comprise one or more baffles that individually completely surround one or more openings though the bottom seal.

Embodiment 17: The cartridge of any of embodiments 1 to 16, comprising baffles that individually completely surround a plurality of air apertures through the bottom seal.

Embodiment 18: The cartridge of any of embodiments 1 to 17, wherein the bottom seal comprises an outer edge that is in contact with an inner surface of an outer wall of the tank body.

Embodiment 19: The cartridge of any of embodiments 1 to 18, wherein the outer edge of the bottom seal comprises a plurality of ribs.

Embodiment 20: The cartridge of any of embodiments 1 to 19, wherein the cartridge includes at least two separate splitting elements effective to split the air stream at least twice while passing through the air pathway.

Embodiment 21: The cartridge of any of embodiments 1 to 20, wherein the at least two separate splitting elements are positioned in the cartridge so that the air stream is split in a direction that is substantially parallel to a longitudinal axis of the cartridge and is split in a direction that is substantially orthogonal to the longitudinal axis of the cartridge.

Embodiment 22: The cartridge of any of embodiments 1 to 21, wherein the atomizer comprises a heater.

Embodiment 23: The cartridge of any of embodiments 1 to 22, wherein the atomizer further comprises a liquid transport element.

Embodiment 24: A cartridge for use in an aerosol delivery device, the cartridge comprising: a body including an outer wall and defining a reservoir, an aerosol passage in fluid communication with an aerosol outlet, and an opening at a bottom end thereof, the opening being defined by the outer wall; and a sealing member positioned within the tank body between the reservoir and the opening at the bottom of the tank body, the sealing member having an outer edge that is in contact with an inner surface of the outer wall of the tank body, wherein the sealing member includes a bottom wall with at least one aperture therethrough, the at least one aperture being surrounded by a baffle that extends upward from the bottom wall.

These and other features, aspects, and advantages of the disclosure will be apparent from a reading of the following detailed description together with the accompanying drawings, which are briefly described below. The disclosure includes any combination of elements, components, and features that are described herein, regardless of whether such elements, components, and features are expressly combined in a specific embodiment description herein. This disclosure is intended to be read holistically such that any separable features, components, or elements of the disclosure, in any of its various aspects and embodiments, should be viewed as intended to be combinable unless the context clearly dictates otherwise.

### BRIEF DESCRIPTION OF THE FIGURES

Having thus described the disclosure in the foregoing general terms, reference will now be made to the accompanying drawings, which are not necessarily drawn to scale, and wherein:

FIG. 1 illustrates a perspective view of an aerosol delivery device, according to example implementations of the present disclosure.

FIG. 2 illustrates a perspective view of a control device of an aerosol delivery device, according to example implementations of the present disclosure.

FIG. 3 illustrates an exploded perspective view of a control device of an aerosol delivery device, according to an example implementation of the present disclosure.

FIG. 4A illustrates a front view of a control device of an aerosol delivery device, according to an example implementation of the present disclosure.

FIG. 4B illustrates a corresponding section view of the control device of FIG. 4A, according to an example implementation of the present disclosure.

FIG. 5A illustrates a side view of a control device of an aerosol delivery device, according to an example implementation of the present disclosure.

FIG. 5B illustrates a corresponding section view of the control device of FIG. 5A, according to an example implementation of the present disclosure.

FIG. 6 illustrates a perspective partial section view of a control device of an aerosol delivery device, according to an example implementation of the present disclosure.

FIG. 7 illustrates an exploded perspective view of a cartridge of an aerosol delivery device, according to an example implementation of the present disclosure.

FIG. 8 illustrates a partial cross-sectional view of a cartridge body according to an example implementation of the present disclosure.

FIG. 9 illustrates a partial cross-sectional view of a cartridge according to an example implementation of the present disclosure.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

As described hereinafter, embodiments of the present disclosure relate to aerosol delivery devices or vaporization devices, said terms being used herein interchangeably. Aerosol delivery devices according to the present disclosure use electrical energy to heat a material (preferably without combusting the material to any significant degree and/or without significant chemical alteration of the material) to form an inhalable substance; and components of such devices have the form of articles that most preferably are sufficiently compact to be considered hand-held devices. That is, use of components of preferred aerosol delivery devices does not result in the production of smoke—i.e., from by-products of combustion or pyrolysis of tobacco, but rather, use of those preferred systems results in the production of vapors resulting from volatilization or vaporization of certain components incorporated therein. In preferred embodiments, components of aerosol delivery devices may be characterized as electronic cigarettes, and those electronic cigarettes most preferably incorporate tobacco and/or components derived from tobacco, and hence deliver tobacco derived components in aerosol form.

Aerosol delivery devices may provide many of the sensations (e.g., inhalation and exhalation rituals, types of tastes or flavors, organoleptic effects, physical feel, use rituals, visual cues such as those provided by visible aerosol, and the like) of smoking a cigarette, cigar, or pipe that is employed by lighting and burning tobacco (and hence inhaling tobacco smoke), without any substantial degree of combustion of any component thereof. For example, the user of an aerosol generating device of the present disclosure can hold and use that piece much like a smoker employs a traditional type of smoking article, draw on one end of that piece for inhalation of aerosol produced by that piece, take or draw puffs at selected intervals of time, and the like.

Aerosol delivery devices of the present disclosure also can be characterized as being vapor-producing articles or medicament delivery articles. Thus, such articles or devices can be adapted so as to provide one or more substances (e.g., flavors and/or pharmaceutical active ingredients) in an inhalable form or state. For example, inhalable substances can be substantially in the form of a vapor (i.e., a substance that is in the gas phase at a temperature lower than its critical point). Alternatively, inhalable substances can be in the form of an aerosol (i.e., a suspension of fine solid particles or liquid droplets in a gas). For purposes of simplicity, the term “aerosol” as used herein is meant to include vapors, gases, and aerosols of a form or type suitable for human inhalation, whether or not visible, and whether or not of a form that might be considered to be smoke-like.

Aerosol delivery devices of the present disclosure most preferably comprise some combination of a power source (i.e., an electrical power source), at least one control component (e.g., means for actuating, controlling, regulating and ceasing power for heat generation, such as by controlling electrical current flow from the power source to other components of the article—e.g., a microcontroller or microprocessor), a heater or heat generation member (e.g., an electrical resistance heating element or other component, which alone or in combination with one or more further elements may be commonly referred to as an “atomizer”), a liquid composition (e.g., commonly an aerosol precursor composition liquid capable of yielding an aerosol upon application of sufficient heat, such as ingredients commonly referred to as “smoke juice,” “e-liquid” and “e-juice”), and a mouthpiece or mouth region for allowing draw upon the aerosol delivery device for aerosol inhalation (e.g., a defined airflow path through the article such that aerosol generated can be withdrawn therefrom upon draw). In some embodiments, an aerosol delivery device can be a power unit or control unit, which unit typically comprises at least a power source and a control component. In some embodiments, an aerosol delivery device can be a cartridge or pod that can typically comprise at least atomizer components, a reservoir suitable for storage of liquid, and a mouthpiece or mouth region. In some embodiments, an aerosol delivery device can be a combination of a power unit or control unit with a cartridge or pod.

More specific formats, configurations and arrangements of components within the aerosol delivery devices of the present disclosure will be evident in light of the further disclosure provided hereinafter. Additionally, the selection and arrangement of various aerosol delivery device components can be appreciated upon consideration of the commercially available electronic aerosol delivery devices, such as those representative products referenced in the background art section of the present disclosure.

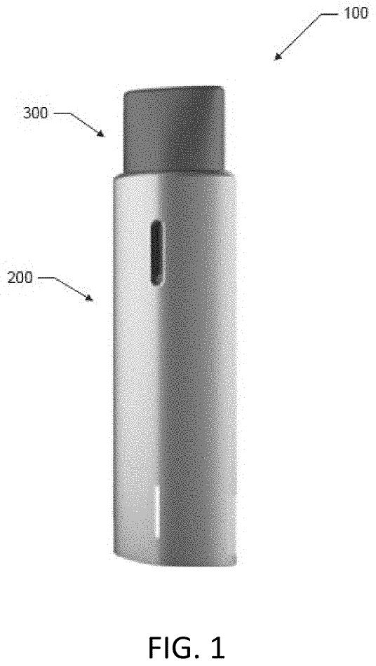

An example implementation of an aerosol delivery device 100 of the present disclosure is shown in FIG. 1. As illustrated, the aerosol delivery device 100 includes a control/power device 200 and a removable cartridge/pod 300. Although only one cartridge is shown in the depicted implementation, it should be understood that, in various implementations, the aerosol delivery device 100 may comprise an interchangeable system. For example, in one or more implementations, a single control device may be usable with a plurality of different cartridges. Likewise, in one or more implementations, a single cartridge may be usable with a plurality of different control devices.

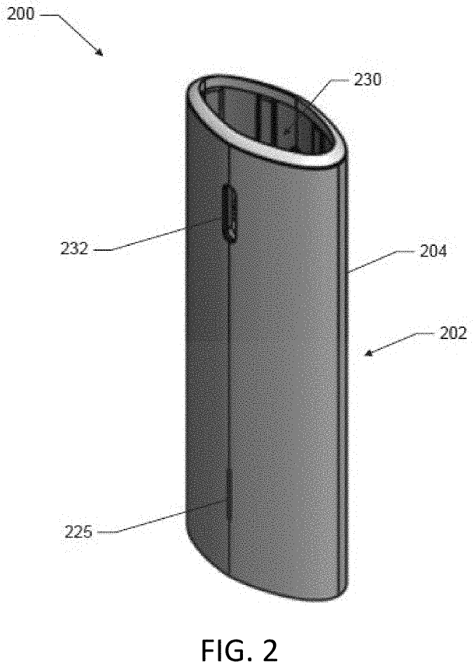

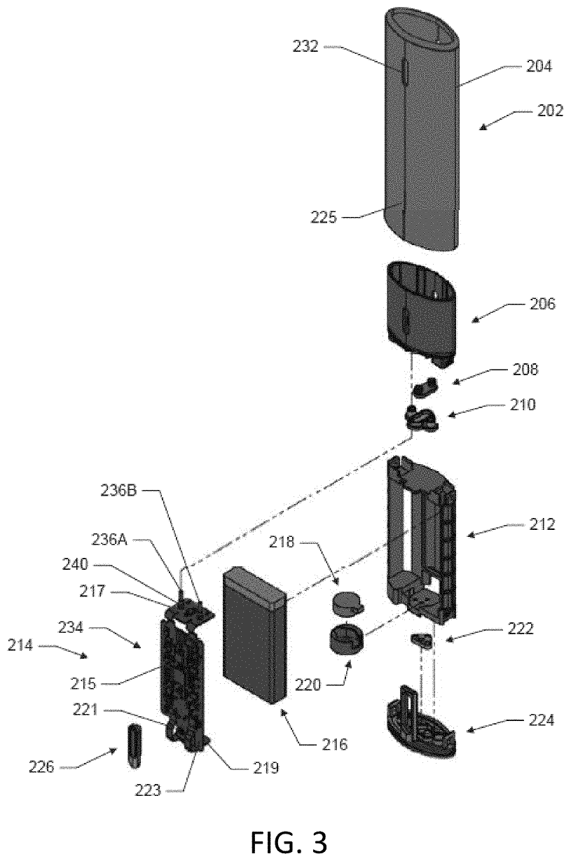

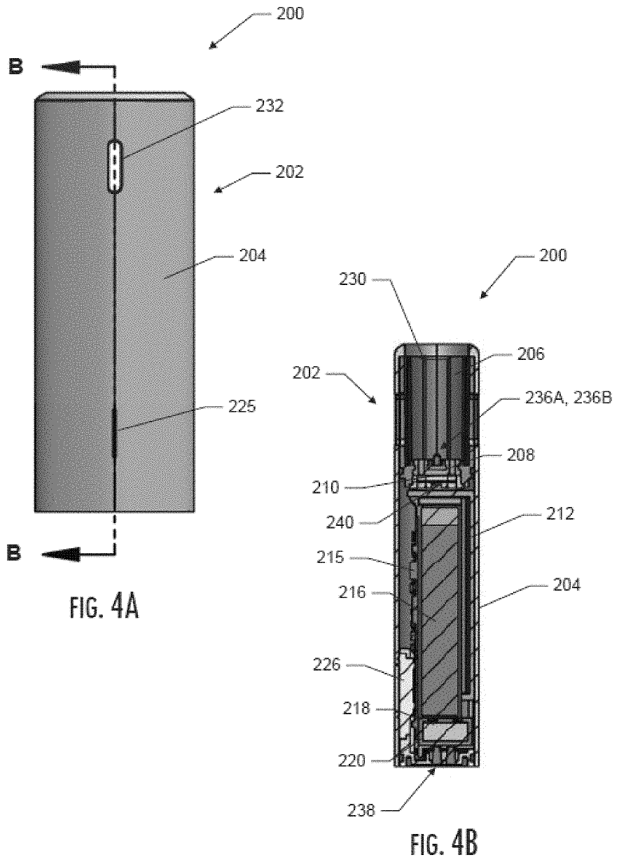

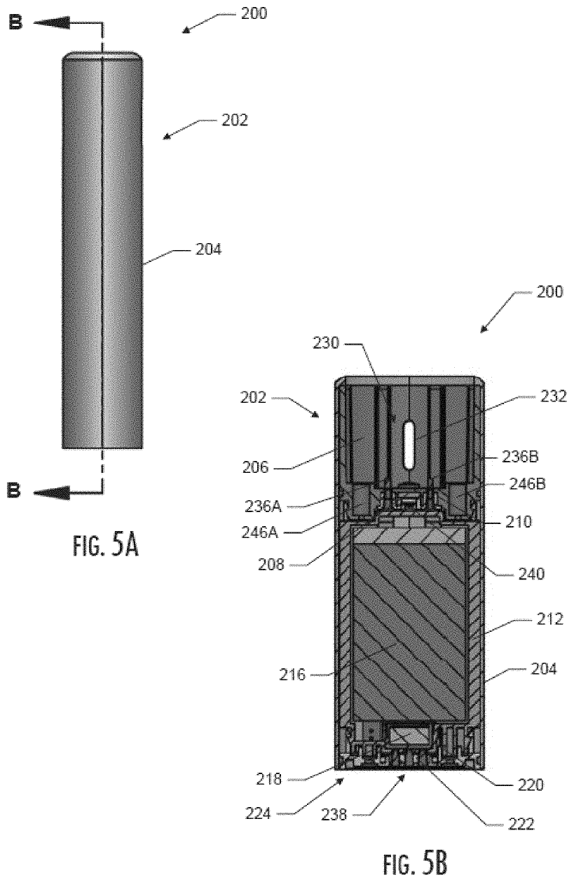

FIG. 2 illustrates a perspective view of a further embodiment of the control device 200, and FIG. 3 illustrates an exploded perspective view thereof. FIG. 4A, FIG. 4B, FIG. 5A, and FIG. 5B illustrate further views of the control device. The figures in total illustrate that the control device 200 can be provided in a variety of form factors while still incorporating similar internal components. Thus, it is understood that the components illustrated in FIG. 2 through FIG. 5B may be utilized with the form factor illustrated in FIG. 1 as well as other form factors that may be envisioned.

As shown in the figures, the control device 200 of the depicted implementation generally includes a housing 202 defining an outer wall 204, an upper frame 206, an upper frame seal 208, a pressure sensor seal 210, a lower frame 212, a control component 214, a battery 216, a vibration motor 218, a motor housing 220, a pin seal 222, an end cap 224, and a light diffuser 226. The upper frame 206 of the control device 200 defines a cartridge receiving chamber 230 within which a cartridge may be coupled. The control device 200 also includes a pair of opposite windows 232 that are defined through the outer wall 204 of the housing 202, as well as through the upper frame 206. In alternative embodiments, the windows 232 may be positioned below the upper frame 206. It thus will be appreciated that the illustrated windows 232 are provided by way of example and not by way of limitation. For example, alternative implementations may include a window having a different shape than that illustrated. As another example, some implementations may include only a single window. In still other implementations, there need not be any windows. In the depicted implementation, the upper frame 206 and the housing 202 represent different parts; however, in other implementations, the upper frame and the housing may be continuously formed such that they comprise the same part.

In the depicted implementation, the housing 202 comprises a metal material, such as, for example, aluminum; however, in other implementations the housing may comprise a metal alloy material, and in still other implementations the housing may comprise a molded plastic material. In the depicted implementation, one or more of the housing 202, upper frame 206, lower frame 212, and end cap 224 may be made of a molded polymer material, such as, for example, a molded plastic material (e.g., polybutylene terephthalate (PBT), acrylonitrile butadiene styrene (ABS), polyethylene, polycarbonate, Polyamide (Nylon), high impact polystyrene, polypropylene, and combinations thereof). In other implementations, one or more of these components may be made of other materials, including, for example, metal materials (e.g., aluminum, stainless steel, metal alloys, etc.), glass materials, ceramic materials (e.g., alumina, silica, mullite, silicon carbide, silicon nitride, aluminum nitride, etc.), composite materials, and/or any combinations thereof.

In the depicted implementation, the lower frame 212 is configured to contain the battery 216 in an interior area thereof. In the depicted implementation, the battery may comprise a lithium polymer (LiPo) battery; however various other batteries may be suitable. Some other examples of batteries that can be used according to the disclosure are described in U.S. Pat. App. Pub. No. 2010/0028766 to Peckerar et al., the disclosure of which is incorporated herein by reference in its entirety. In some implementations, other types of power sources may be utilized. For example, in various implementations a power source may comprise a replaceable battery or a rechargeable battery, solid-state battery, thin-film solid-state battery, rechargeable supercapacitor or the like, and thus may be combined with any type of recharging technology, including connection to a wall charger, connection to a car charger (e.g., cigarette lighter receptacle, USB port, etc.), connection to a computer, such as through a universal serial bus (USB) cable or connector (e.g., USB 2.0, 3.0, 3.1, USB Type-C), connection to a USB connector (e.g., USB 2.0, 3.0, 3.1, USB Type-C as may be implemented in a wall outlet, electronic device, vehicle, etc.), connection to a photovoltaic cell (sometimes referred to as a solar cell) or solar panel of solar cells, a wireless charger, such as a charger that uses inductive wireless charging (including for example, wireless charging according to the Qi wireless charging standard from the Wireless Power Consortium (WPC)), or a wireless radio frequency (RF) based charger, and connection to an array of external cell(s) such as a power bank to charge a device via a USB connector or a wireless charger. An example of an inductive wireless charging system is described in U.S. Pat. App. Pub. No. 2017/0112196 to Sur et al., which is incorporated herein by reference in its entirety. In further implementations, a power source may also comprise a capacitor. Capacitors are capable of discharging more quickly than batteries and can be charged between puffs, allowing the battery to discharge into the capacitor at a lower rate than if it were used to power the heating member directly. For example, a supercapacitor—e.g., an electric double-layer capacitor (EDLC)— may be used separate from or in combination with a battery. When used alone, the supercapacitor may be recharged before each use of the article. Thus, the device may also include a charger component that can be attached to the smoking article between uses to replenish the supercapacitor. Examples of power supplies that include supercapacitors are described in U.S. Pat. App. Pub. No. 2017/0112191 to Sur et al., which is incorporated herein by reference in its entirety.

The aerosol delivery device 100 of the depicted implementation includes a control mechanism in the form of the control component 214, which is configured, in part, to control the amount of electric power provided to the heating member of the cartridge 300. Although other configurations are possible, the control component 214 of the depicted implementation comprises a circuit board 234 (e.g., a printed circuit board (PCB)) that includes both rigid and flexible portions. In particular, the circuit board 234 of the depicted implementation includes a rigid central section 215 and two rigid end sections comprising a proximal end section 217 and a distal end section 219, with each of the end sections 217, 219 being connected to the central section 215 by a respective flexible connection. In such a manner, when the lower frame 212, battery 216, and circuit board 234 are assembled into the control device 200, the central section 215 of the circuit board 234 is configured to be disposed proximate a major surface of the battery 216, and the two end sections 217, 219 are configured to be disposed substantially perpendicular to the central section 215 (e.g., “substantially” indicating exactly perpendicular or within +/−5 degrees, 4 degrees, 3 degrees, 2 degrees, or 1 degree of exactly perpendicular). In particular, the proximal end section 217 of the circuit board 234 is configured to extend over the top of the lower frame 212, and the distal end section 219 is configured to extend over the bottom of the lower frame 212. The lower frame 212 of the control device 200 is also configured to contain the motor housing 220, into which the vibration motor 218 is received. In various implementations, the vibration motor 218 may provide haptic feedback relating to various operations of the device 100.

The central section 215 of the depicted implementation also includes an indicator in the form of a light source 221. In some implementations, the light source may comprise, for example, at least one light emitting diode (LED) capable of providing one or more colors of light. In other implementations, the light source may be configured to illuminate in only one color, while in other implementations, the light source may be configured to illuminate in variety of different colors. In still other implementations, the light source may be configured to provide white light. In the depicted implementation, the light source 221 comprises an RGB (red, green, blue) LED that is configured to provide a variety of colors of light, including white light. The central section 215 of the depicted circuit board 234 also includes electrical contacts 223 that are configured to operatively connect the circuit board 234 to the vibration motor 218. Other types of electronic components, structures and configurations thereof, features thereof, and general methods of operation thereof, are described in U.S. Pat. No. 4,735,217 to Gerth et al.; U.S. Pat. No. 4,947,874 to Brooks et al.; U.S. Pat. No. 5,372,148 to McCafferty et al.; U.S. Pat. No. 6,040,560 to Fleischhauer et al.; U.S. Pat. No. 7,040,314 to Nguyen et al. and U.S. Pat. No. 8,205,622 to Pan; U.S. Pat. App. Pub. Nos. 2009/0230117 to Fernando et al., 2014/0060554 to Collet et al., and 2014/0270727 to Ampolini et al.; and U.S. Pat. App. Pub. No. 2015/0257445 to Henry et al.; which are incorporated herein by reference. Yet other features, controls or components that can be incorporated into aerosol delivery devices of the present disclosure are described in U.S. Pat. No. 5,967,148 to Harris et al.; U.S. Pat. No. 5,934,289 to Watkins et al.; U.S. Pat. No. 5,954,979 to Counts et al.; U.S. Pat. No. 6,040,560 to Fleischhauer et al.; U.S. Pat. No. 8,365,742 to Hon; U.S. Pat. No. 8,402,976 to Fernando et al.; U.S. Pat. App. Pub. Nos. 2010/0163063 to Fernando et al.; 2013/0192623 to Tucker et al.; 2013/0298905 to Leven et al.; 2013/0180553 to Kim et al., 2014/0000638 to Sebastian et al., 2014/0261495 to Novak et al., and 2014/0261408 to DePiano et al.; which are incorporated herein by reference in their entireties.

In the depicted implementation, the light source 221 is covered by the light diffuser 226, a portion of which is configured to be received by the end cap 224. In such a manner, when assembled, the light diffuser 226 is positioned in or proximate an aperture 225 defined in the outer wall 204 of the housing 202 and proximate a distal end thereof. In the depicted implementation, the aperture 225 comprises a narrow, elongate opening; however, in other implementations, the aperture may be provided in any desired shape and may be positioned at any location on the control device 200. In some implementations, the light diffuser 226 may comprise a transparent or translucent member configured to allow a user to view the light source 221 from the outside of the housing 202. In the depicted implementation, the light diffuser 226 may be made of a molded polymer material, such as, for example, a molded plastic material (e.g., acrylonitrile butadiene styrene (ABS), polyethylene, polycarbonate, Polyamide (Nylon), high impact polystyrene, polypropylene, and combinations thereof), although other materials, including glass, are possible. In various implementations, further indicators (e.g., other haptic feedback components, an audio feedback component, or the like) can be included in addition to or as an alternative to the indicators included in the depicted implementation. Additional representative types of components that yield visual cues or indicators, such as LED components, and the configurations and uses thereof, are described in U.S. Pat. No. 5,154,192 to Sprinkel et al.; U.S. Pat. No. 8,499,766 to Newton and U.S. Pat. No. 8,539,959 to Scatterday; U.S. Pat. App. Pub. No. 2015/0020825 to Galloway et al.; and U.S. Pat. App. Pub. No. 2015/0216233 to Sears et al.; which are incorporated herein by reference in their entireties. While FIG. 1 through and FIG. 3 illustrate both a window 232 and an aperture 225, it is understood that only one or the other may be present. For example, only aperture 225 may be present. Thus, it is understood that the light source 221 and any other appropriate component of the control device 200 may be positioned according to the form factor of the housing 202.

Although other configurations are possible, the proximal end section 217 of the circuit board 234 of the depicted implementation includes a pair of conductive pins 236A, 236B, as well as a pressure sensor 240. In the depicted implementation, the conductive pins 236A, 236B comprise spring-loaded pins (e.g., electrical pogo pins) that extend through the upper frame 206 such that portions of the ends of the pins 236A, 236B extend into the cartridge receiving chamber 230 and are biased in that position due to the force of the internal springs of the conductive pins 236A, 236B. In such a manner, when a cartridge is coupled with the control device 200, the conductive pins 236A, 236B are configured to contact corresponding features of the cartridge and deflect downward (e.g., toward the lower frame 212) against the force of the springs, thus operatively connecting the installed cartridge with the control component 214 and the battery 216. In the depicted implementation, the conductive pins 236A, 236B comprise gold plated metal pins; however, other materials or combinations of materials, which may also include coatings and/or platings of electrically conductive materials, are possible. Examples of electrically conductive materials, include, but are not limited to, copper, aluminum, platinum, gold, silver, iron, steel, brass, bronze, graphite, conductive ceramic materials, and/or any combination thereof. Although other profiles are possible, the ends of the conductive pins 236A, 236B of the depicted implementation have a rounded profile such that deflection of the conductive pins 236A, 236B is facilitated when a cartridge is inserted into the cartridge receiving chamber 230. In other implementations, the conductive pins may be positioned in other locations of the cartridge receiving chamber 230, such as, for example, proximate the top of the cartridge receiving chamber 230. In other implementations, the conductive pins may be positioned at a point on the sides of the upper frame 206 between the proximal end of the outer housing 202 and the bottom wall of the upper frame 206. Further, in still other implementations the conductive pins may be positioned between a midpoint of the sidewalls and the proximal end of the outer housing 202 (i.e., in an upper half of the sidewalls). Alternatively, the conductive pins may be positioned between a midpoint of the sidewalls and the bottom wall of the inner frame wall (e.g., in a lower half of the sidewalls). Moreover, in still other implementations, the conductive pins may be present at any position of the upper frame 206.

In various implementations, the aerosol delivery device 100 may include an airflow sensor, pressure sensor, or the like. As noted above, the control component 214 of the depicted implementation includes a pressure sensor 240, which is positioned proximate and below the cartridge receiving chamber 230. The position and function of the pressure sensor 240 of the depicted implementation will be described below; however, in other implementations an airflow or pressure sensor may be positioned anywhere within the control device 200 so as to subject to airflow and/or a pressure change that can signal a draw on the device and thus cause the battery 216 to delivery power to the heating member of the cartridge 300. Various configurations of a printed circuit board and a pressure sensor, for example, are described in U.S. Pat. Pub. No. 2015/0245658 to Worm et al., the disclosure of which is incorporated herein by reference in its entirety. In the absence of an airflow sensor, pressure sensor, or the like, an aerosol delivery device may be activated manually, such as via a pushbutton that may be located on the control device and/or the cartridge. For example, one or more pushbuttons may be used as described in U.S. Pat. App. Pub. No. 2015/0245658 to Worm et al., which is incorporated herein by reference in its entirety. Likewise, a touchscreen may be used as described in U.S. patent application Ser. No. 14/643,626, filed Mar. 10, 2015, to Sears et al., which is incorporated herein by reference in its entirety. As a further example, components adapted for gesture recognition based on specified movements of the aerosol delivery device may be used as an input. See U.S. Pat. App. Pub. No. 2016/0158782 to Henry et al., which is incorporated herein by reference in its entirety.

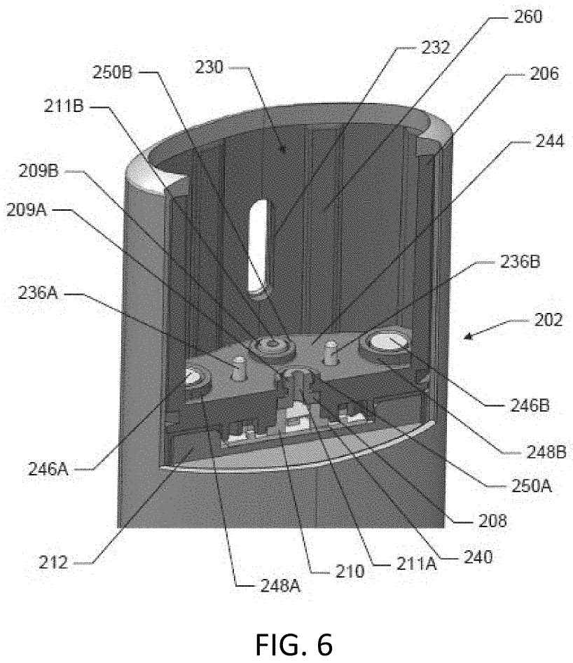

Although not included in the depicted implementation, some implementations may include other types of input elements, which may replace or supplement an airflow or pressure sensor. The input may be included to allow a user to control functions of the device and/or for output of information to a user. Any component or combination of components may be utilized as an input for controlling the function of the device. In some implementations, an input may comprise a computer or computing device, such as a smartphone or tablet. In particular, the aerosol delivery device may be wired to the computer or other device, such as via use of a USB cord or similar protocol. The aerosol delivery device may also communicate with a computer or other device acting as an input via wireless communication. See, for example, the systems and methods for controlling a device via a read request as described in U.S. Pat. App. Pub. No. 2016/0007561 to Ampolini et al., the disclosure of which is incorporated herein by reference in its entirety. In such embodiments, an APP or other computer program may be used in connection with a computer or other computing device to input control instructions to the aerosol delivery device, such control instructions including, for example, the ability to form an aerosol of specific composition by choosing the nicotine content and/or content of further flavors to be included. Additional representative types of sensing or detection mechanisms, structure and configuration thereof, components thereof, and general methods of operation thereof, are described in U.S. Pat. No. 5,261,424 to Sprinkel, Jr.; U.S. Pat. No. 5,372,148 to McCafferty et al.; and PCT WO 2010/003480 to Flick; which are incorporated herein by reference in their entireties. In the depicted implementation, the pressure sensor seal 210 is configured to cover the pressure sensor 240 to protect it from any liquid and/or aerosol from an installed cartridge. In addition, the pressure sensor seal 210 of the depicted implementation is configured to seal the conductive pins 236A, 236B. In such a manner, the pressure sensor seal 210 of the depicted implementation may be made of silicone rubber, boron nitride (BN) rubber, natural rubber, thermoplastic polyurethane, or another resilient material. In the depicted implementation, the upper frame seal 208 is configured to be positioned proximate and above the pressure sensor seal 210, such that a pair of upper frame seal tubes 209A, 209B (see FIG. 6) of the upper frame seal 208 extend through the upper frame 206 and into the cartridge receiving chamber 230. The upper frame seal 208 of the depicted implementation may also be made of a silicone, thermoplastic polyurethane, or another resilient material.

Although other configurations are possible, the distal end section 219 of the circuit board 234 includes the external connection element 238. In various implementations, the external connection element 238 may be configured for connecting to an external connector and/or a docking station or other power or data source. For example, in some implementations an external connector may comprise first and second connector ends that may be interconnected by a union, which may be, for example, a cord of variable length. In some implementations, the first connector end may be configured for electrical and, optionally, mechanical connection with the device (100,200), and the second connector end may be configured for connection to a computer or similar electronic device or for connection to a power source. An adaptor including a USB connector at one end and a power unit connector at an opposing end is disclosed in U.S. Pat. App. Pub. No. 2014/0261495 to Novak et al., which is incorporated herein by reference in its entirety. In the depicted implementation, the pin seal 222 is configured to seal the interface between the external connection element 238 and the end cap 224. In such a manner, the pin seal 222 of the depicted implementation may be made of a silicone, thermoplastic polyurethane, or another resilient material. In the depicted implementation, one or more pins of the external connection element 238 may extend through the end cap 224 of the control device as noted above.

In various implementations, the control device may include one or more components configured to meet battery outgassing requirements under UL 8139. For example, the control device may include an end cap configured to eject in the event that sudden pressurization occurs within the control device enclosure. In one implementation, the end cap may include retaining pins that extend substantially perpendicularly from a wall of the end cap (with “substantially” having a meaning as already described above). The retaining pins may be configured to mate with receiving features (e.g., holes) in a frame of the control device to establish a friction fit or press fit that may be overcome if an internal pressure within the control device housing exceeds a defined internal pressure.

FIG. 6 illustrates a perspective partial section view of a control device of an aerosol delivery device. In particular, FIG. 6 illustrates a partial section view of the housing 202, upper frame 206, upper frame seal 208, pressure sensor seal 210, pressure sensor 240, and lower frame 212 of the control device 200. As shown in the figure, a portion of the conductive pins 236A, 236B of the control component 214 extend through the upper frame 206. In particular, a portion of the conductive pins 236A, 236B of the depicted implementation, which as noted above comprise spring-loaded contacts, extend through a recessed surface 244 of the upper frame 206 and into the cartridge receiving chamber 230. In addition, a portion of the upper frame seal tubes 209A, 209B (which define respective seal tube channels 211A, 211B) of the upper frame seal 208 extend through the upper frame 206 and are exposed in the cartridge receiving chamber 230. As will be described in more detail below, regardless of the orientation of an installed cartridge, the conductive pins 236A, 236B and one of the upper frame seal tubes 209A, 209B are configured to substantially align with corresponding features of an installed cartridge. It is understood, however, that the positions of these components and the number of each component included in the control device can vary as desired to substantially match a cartridge as further defined herein.

As also shown in the figure, the upper frame 206 includes a pair of magnets 246A, 246B that are also exposed in the cartridge receiving chamber 230. In various implementations, the magnets 246A, 246B may comprise any type of magnets, including rare earth magnets. For example, in some implementations, one or more of the magnets may comprise Neodymium magnets (also known as NdFeB, NIB, or Neo magnets). In various implementations, different grades of Neodymium magnets may be used, including, for example, N35, N38, N40, N42, N45, N48, N50, and/or N52 grades. In other implementations, one or more of the magnets may comprise Samarium Cobalt magnets (also known as SmCo magnets). In still other implementations, one or more of the magnets may comprise Ceramic/Ferrite magnets. In other implementations, one or more of the magnets may comprise Aluminum-Nickel-Cobalt (AlNiCo) magnets. In any of the foregoing implementations, one or more of the magnets may be plated and/or coated. For example, in some implementations, one or more of the magnets may be coated with nickel. In other implementations, one or more magnets may be coated with one or more of zinc, tin, copper, epoxy, silver and/or gold. In some implementations, one or more of the magnets may be coated with combinations of these materials. For example, in one implementation, one or more of the magnets may be coated with nickel, copper, and nickel again. In another implementation, one or more of the magnets may be coated with nickel, copper, nickel, and a top coating of gold.

In the depicted implementation, each magnet 246A, 246B is substantially surrounded by a respective location feature 248A, 248B of the upper frame 206, wherein the location features 248A, 248B also extend into the cartridge receiving chamber 230. Likewise, each upper frame seal tube 209A, 209B of the upper frame seal 208 is substantially surrounded by a respective location feature 250A, 250B. As will be discussed in more detail below, one or more of the location features 248A, 248B, 250A, 250B of the upper frame 206 are configured as stopping or vertical locating features for an installed cartridge and are thus configured to position the cartridge 300 with respect to the recessed surface 244 of the upper frame 206 of the control device 200. It is again understood that the position and/or number of magnets and corresponding components in the control device may vary.

As noted above, a portion of the cartridge 300 is configured to be coupled with the cartridge receiving chamber 230 of the inner frame 206 of the control device 200 such that mechanical and electrical connections are created between the cartridge 300 and the control device 200. In particular, when the cartridge 300 of the depicted implementation is coupled with the upper frame 206 of the control device 200, a magnetic connection is created between the magnets 246A, 246B located in the upper frame 206 and corresponding features of the cartridge 300. In addition, when the cartridge 300 of the depicted implementation is coupled with the inner frame 206, an electrical connection is created between the pair conductive pins 236A, 236B of the control device 200 and corresponding features of the cartridge 300. As such, when the cartridge 300 is received in the receiving chamber 230 of the control device 200, the cartridge 300 may be operatively connected to the control component 214 and the battery 216 of the control device 200. Thus, when the cartridge 300 of the depicted implementation is coupled with the control device 200, the cartridge 300 is mechanically biased into connection with the control device 200 such that electrical connection is maintained between the cartridge and the control device. It should be understood that for the purposes of the present disclosure, the term “operatively connected” and other related forms thereof should be interpreted broadly so as to encompass components that are directly connected and/or connected via one or more additional components.

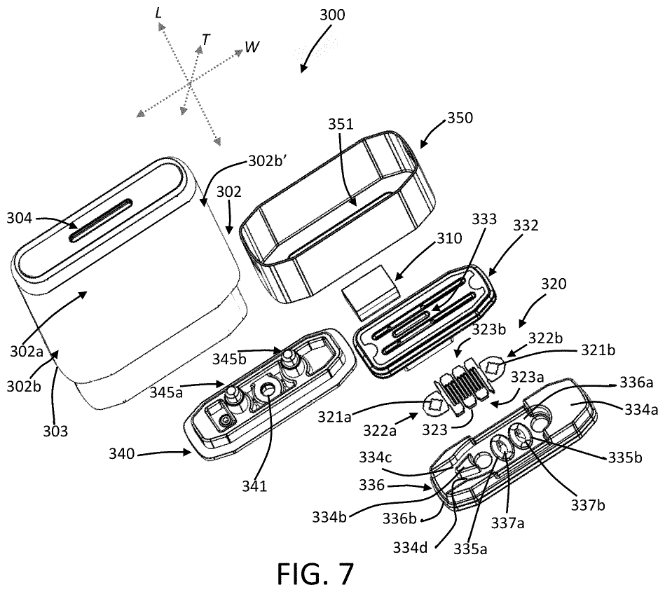

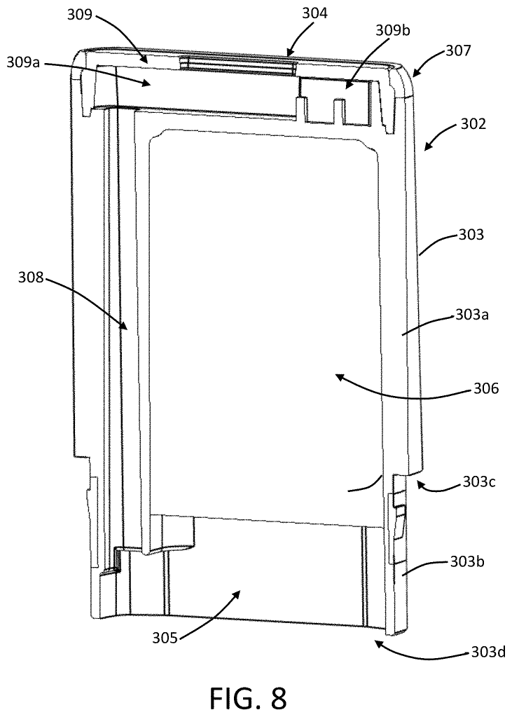

FIG. 7 illustrates an exploded view of the cartridge. Although other configurations are possible, the cartridge 300 of the depicted implementation generally includes a tank body 302 with an aerosol outlet 304 formed in a mouthend thereof. FIG. 8 shows a cross-sectional view of the tank body 302. The term “tank body” is not intended to be limiting, and it is understood that the term “body” can be used interchangeably with the term “tank body” without departing from the original use of the term. Likewise, other components referenced herein as being relative to a tank (e.g., a “tank wall”, etc.) are intended to be non-limiting. The word “tank” is thus used to provide reference and not to limit structure.

As seen in the figures, the tank body 302 defines a function chamber 305 at an end opposite from the mouthend, and further components of the cartridge can be positioned therein as further described below. The tank body 302 further defines a reservoir 306 wherein a liquid may be stored. The reservoir 306 may be substantially an open space (“substantially indicating that minor, and particularly non-functional, amounts of components may be present, or the space may be completely devoid of anything apart from the liquid to be stored therein); however, if desired, a substrate (e.g., a woven or nonwoven fabric) may be positioned therein to assist in retaining liquid within the reservoir. An aerosol pathway 308 is defined along one side of the tank body 302. As further discussed below, vapor formed by the atomizer components in the function chamber 305 can mix with air to form an aerosol, which can be drawn through the aerosol pathway 308 and exit the mouthend of the tank body 302 through the aerosol outlet 304. If desired, in some embodiments, a plurality of aerosol pathways maybe included in the tank body 302. In preferred embodiments, however, it can be desirable to include only a single aerosol pathway, and additional aerosol pathway(s) can be expressly excluded. As such, the aerosol pathway 308 can be offset from the reservoir 306. The tank body 302 can include an outlet plug 309 that more particularly defines the aerosol outlet 304. The outlet plug can function with internal features of the tank body 302 to define an outlet chamber 309a that includes one or more baffles 309b. The one or more baffles 309b can function to trap any condensed liquid to resist or prevent leaking of liquid from the aerosol outlet 304.

In the depicted implementation, the tank body 302 and/or the outlet plug may be made of a molded polymer material, such as, for example, a molded plastic material (e.g., polypropylene, acrylonitrile butadiene styrene (ABS), polyethylene, polycarbonate, Polyamide (Nylon), high impact polystyrene, polyesters (including copolyesters, such as Tritan™ polymer), and combinations thereof). Other materials, however, are not necessarily excluded.

The tank body 302 may be injection molded or may be prepared using injection molding in combination with further processing steps, including sonic welding, gluing, or otherwise combining multiple elements to form the finished part. In some embodiments, the tank body may be formed utilizing a plurality of injection molding steps so that added components may be combined to form the final unit. For example, the outlet plug 309 may be a separate component that can be insert molded into the tank body 302. In certain embodiments of forming the tank body, a first molding injection can be utilized to form a majority of the tank body 302, including internal features thereof. The first injection molding, if desired, can be done with a first polymer material having defined characteristics, including but not limited to being either transparent or translucent or being substantially opaque or colored. A second injection molding can then be carried out using a second polymer material that differs from the first polymer material in relation to composition and/or in relation to certain defined characteristics, including but not limited to being either transparent or translucent or being substantially opaque or colored. The outlet plug 309 can be inserted between the first molding injection and the second molding injection to form a mouthpiece 307 at the mouthend of the tank body 302. Beneficially, in some embodiments, the tank body 302 thus can be prepared without need of post processing, such as welding, gluing, or manual assembly. The dual injection steps thus allow for formation of one or more chambers within the tank body with ease of processing. Likewise, the dual injection steps can be beneficial to provide the tank body with different characteristics in different sections thereof. As otherwise described herein, for example, the dual injection steps can be carried out using polymeric materials of different light transmission properties. For example, the polymer used in the first injection can be substantially transparent or translucent, and the polymer used in the second injection can be substantially opaque or colored. The second injection can cover portions of the tank body 302 formed in the first injection, and the finished tank body can thus have transparent or translucent portions while also having opaque or colored portions.

A wall 303 of the tank body 302 can have differing thicknesses along the length of the tank body. For example, a body section 303a of the tank wall may define a greater wall thickness than a connecting section 303b of the tank wall. The different thicknesses then may form a ledge 303c (or flange) that can substantially surround the tank body 302. The ledge 303c may define an insert depth by which the cartridge 300 may be inserted into a control device 200. Likewise, the different thicknesses may account at least in part for placement of the bottom cap 350 on the tank body 302. In some embodiments, the injection molding described above may also account at least in part for differences in thickness of the tank body. The second injection molding may cover all or a portion of the body section 303a of the tank wall, may cover all or a portion of the connecting section 303b of the tank wall, or may cover all or a portion of both of the body section and the connecting section. As an example, the second injection molding may cover a portion of the body section and a portion of the connecting section.

With reference to FIG. 7, the tank body 302 may be defined in some embodiments in relation to one or more of a length measured along a longitudinal axis L, a width measured along a transverse axis W that is transverse to the longitudinal axis, and a thickness that is measured along a perpendicular axis T that is perpendicular to the transverse axis. In some embodiments, the length and width of the tank body 320 may both be greater than the thickness of the tank body. The tank body 302 thus may be characterized as including a first pair of wall sections (302a, 302a′) that are substantially front and rear facing and a second pair of wall sections (302b, 302b′) that are substantially side facing. As the cartridge 300 may be reversible in some embodiments, the terms “front” and “rear” may be applied merely to differentiate the two sections of the tank body 302. In embodiments where the cartridge 300 is not reversible and thus may be only inserted into the control body 200 in one orientation, the terms “front” and “rear” may more particularly define the correct orientation of the cartridge 300 relative to the control body 200. The injection molding described above may be specifically applied in relation to the wall sections just described. For example, the first injection molding may substantially define the tank body, and the second injection molding may cover portions of the first pair of wall sections (302a, 302a′) without covering any of the second pair of wall sections (302b, 302b′).

In the depicted implementation, the tank wall 303 can be configured to be transparent or translucent so that the liquid composition contained therein may be visible externally. Alternatively, in some implementations, only a portion or portions of the tank wall may be transparent or translucent while the remaining portion(s) of the tank wall may be substantially opaque. In further implementations, all of the tank wall or one or more portions of the tank wall may be colored. In some implementations, opacity and/or color on the tank body 302 can be configured so that one of the body section 303a and the connecting section 303b is transparent or translucent and the other of the body section and the connecting section is opaque or colored. In an example embodiment, all or a portion of one or both of the substantially front and rear facing wall sections (302a, 302a′) may be opaque (or colored), and all or a portion of one or both of the substantially side facing wall sections (302b, 302b′) may be transparent or translucent. Alternatively, such configuration may be reversed.

As noted above, the reservoir 306 can be configured for storing a liquid, and the liquid can be an aerosol precursor composition—i.e., any liquid that can be converted to a vapor utilizing appropriate atomizing components. For aerosol delivery systems that are characterized as electronic cigarettes, the aerosol precursor composition may incorporate tobacco or components derived from tobacco. In one regard, the tobacco may be provided as parts or pieces of tobacco, such as finely ground, milled or powdered tobacco lamina Tobacco beads, pellets, or other solid forms may be included, such as described in U.S. Pat. App. Pub. No. 2015/0335070 to Sears et al., the disclosure of which is incorporated herein by reference. In another regard, the tobacco may be provided in the form of an extract, such as a spray dried extract that incorporates many of the water soluble components of tobacco. Alternatively, tobacco extracts may have the form of relatively high nicotine content extracts, which extracts also incorporate minor amounts of other extracted components derived from tobacco. In another regard, components derived from tobacco may be provided in a relatively pure form, such as certain flavoring agents that are derived from tobacco. In one regard, a component that is derived from tobacco, and that may be employed in a highly purified or essentially pure form, is nicotine (e.g., pharmaceutical grade nicotine).

In the depicted implementation, the liquid composition, sometime referred to as an aerosol precursor composition or a vapor precursor composition or “e-liquid”, may comprise a variety of components including, by way of example, a polyhydric alcohol (e.g., glycerin, propylene glycol, or a mixture thereof), nicotine, tobacco, tobacco extract, and/or flavorants. Representative types of aerosol precursor components and formulations also are set forth and characterized in U.S. Pat. No. 7,217,320 to Robinson et al. and U.S. Pat. App. Pub. Nos. 2013/0008457 to Zheng et al.; 2013/0213417 to Chong et al.; 2014/0060554 to Collett et al.; 2015/0020823 to Lipowicz et al.; and 2015/0020830 to Koller, as well as WO 2014/182736 to Bowen et al., the disclosures of which are incorporated herein by reference in their entireties. Other aerosol precursors that may be employed include the aerosol precursors that have been incorporated in VUSE® products by R. J. Reynolds Vapor Company, the BLU™ products by Fontem Ventures B.V., the MISTIC MENTHOL product by Mistic Ecigs, MARK TEN products by Nu Mark LLC, the JUUL product by Juul Labs, Inc., and VYPE products by CN Creative Ltd. Also desirable are the so-called “smoke juices” for electronic cigarettes that have been available from Johnson Creek Enterprises LLC. Still further example aerosol precursor compositions are sold under the brand names BLACK NOTE, COSMIC FOG, THE MILKMAN E-LIQUID, FIVE PAWNS, THE VAPOR CHEF, VAPE WILD, BOOSTED, THE STEAM FACTORY, MECH SAUCE, CASEY JONES MAINLINE RESERVE, MITTEN VAPORS, DR. CRIMMY'S V-LIQUID, SMILEY E LIQUID, BEANTOWN VAPOR, CUTTWOOD, CYCLOPS VAPOR, SICBOY, GOOD LIFE VAPOR, TELEOS, PINUP VAPORS, SPACE JAM, MT. BAKER VAPOR, and JIMMY THE JUICE MAN.

The amount of aerosol precursor that is incorporated within the aerosol delivery system is such that the aerosol generating piece provides acceptable sensory and desirable performance characteristics. For example, it is highly preferred that sufficient amounts of aerosol forming material (e.g., glycerin and/or propylene glycol), be employed in order to provide for the generation of a visible mainstream aerosol that in many regards resembles the appearance of tobacco smoke. The amount of aerosol precursor within the aerosol generating system may be dependent upon factors such as the number of puffs desired per aerosol generating piece. As non-limiting examples, a reservoir may be configured to hold about 1 ml or more, about 2 ml or more, about 5 ml or more, or about 10 ml or more of the aerosol precursor composition.

In some implementations, the liquid composition may include one or more flavorants. As used herein, reference to a “flavorant” refers to compounds or components that can be aerosolized and delivered to a user and which impart a sensory experience in terms of taste and/or aroma. Example flavorants include, but are not limited to, vanillin, ethyl vanillin, cream, tea, coffee, fruit (e.g., apple, cherry, strawberry, peach and citrus flavors, including lime and lemon), maple, menthol, mint, peppermint, spearmint, wintergreen, nutmeg, clove, lavender, cardamom, ginger, honey, anise, sage, rosemary, hibiscus, rose hip, yerba mate, guayusa, honeybush, rooibos, yerba santa, bacopa monniera, gingko biloba, withania somnifera, cinnamon, sandalwood, jasmine, cascarilla, cocoa, licorice, and flavorings and flavor packages of the type and character traditionally used for the flavoring of cigarette, cigar, and pipe tobaccos. Syrups, such as high fructose corn syrup, also can be employed. Example plant-derived compositions that may be suitable are disclosed in U.S. Pat. No. 9,107,453 and U.S. Pat. App. Pub. No. 2012/0152265 both to Dube et al., the disclosures of which are incorporated herein by reference in their entireties. The selection of such further components are variable based upon factors such as the sensory characteristics that are desired for the smoking article, and the present disclosure is intended to encompass any such further components that are readily apparent to those skilled in the art of tobacco and tobacco-related or tobacco-derived products. See, e.g., Gutcho, Tobacco Flavoring Substances and Methods, Noyes Data Corp. (1972) and Leffingwell et al., Tobacco Flavoring for Smoking Products (1972), the disclosures of which are incorporated herein by reference in their entireties. It should be understood that reference to a flavorant should not be limited to any single flavorant as described above, and may, in fact, represent a combination of one or more flavorants.

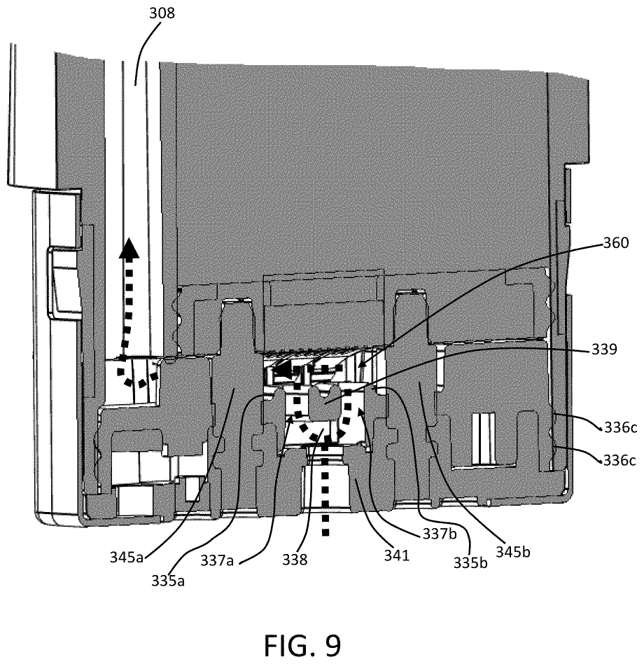

Returning to FIG. 7, the cartridge 300 further can include a liquid transport element 310, a heater 320, and upper frame member 332, a bottom seal 336, and a lower frame member 340. The heater 320 is provided as an example embodiment of an atomizer, and other atomizers may be utilized alternatively, or additionally, to the heater. These elements, when combined, can be positioned in the function chamber 305. The bottom seal 336 of the depicted implementation can be configured to form a substantially liquid tight seal between a lower portion of the tank body 302 and the lower frame member 340. “Substantially” liquid tight can include completely liquid tight or allow for minor deviations due to manufacturing or the like. As such, the bottom seal 336 is positioned between the reservoir 306 and the opening 303d at the bottom end of the tank body 302. The bottom seal includes an outer edge 336b that can be in contact with an inner surface of the outer wall 303 of the tank body 302 when assembled. As seen more clearly in FIG. 9, the outer edge 336b of the bottom seal 336 can include one or a plurality of ribs 336c that extend outward from the outer edge. The rib(s) can compress when in contact with the wall 303 of the tank body 302 to ensure liquid-tight seal. In various implementations, the bottom seal 336 may be made of silicone rubber, boron nitride (BN) rubber, natural rubber, thermoplastic polyurethane, or another resilient material. In the depicted implementation, the lower frame member 340 may be made of a molded polymer material, such as, for example, a molded plastic material (e.g., acrylonitrile butadiene styrene (ABS), polyethylene, polycarbonate, Polyamide (Nylon), high impact polystyrene, polypropylene, and combinations thereof), although other materials are possible. The lower frame member 340 and the bottom seal 336 of the depicted implementation both can include a plurality of slots that are configured to provide air entry into the cartridge 300 and also allow for the electrical contacts (345a, 345b) to project through the lower frame member 340 and the bottom seal 336 to be in electrical contact with the heater 320.

Air entry into the cartridge 300 is further illustrated in FIG. 9. In particular, the lower frame member 340 can include a single air entry 341 (or air inlet). As illustrated, the air entry 341 is substantially centralized (e.g., exactly centralized or within a tolerance range as otherwise described herein) in the lower frame member 340, but other arrangements are not necessarily excluded. When assembled, the bottom seal 336 sits atop the lower frame member 340, and the bottom seal include two air apertures (337a, 337b), which can be substantially symmetrically arranged relative to the air entry 341 in the lower frame member 340. In one or more embodiments, the bottom seal 336 can define a splitting chamber 338 in the lower portion thereof below the two air apertures. The splitting chamber in particular can be defined between the lower frame member 340 and the bottom seal 336 so as to be bounded by a top, planar surface surrounding the air entry 341 and a bottom, planar surface a central member 339 between the two air apertures (337a, 337b) of the bottom seal 336. Air (see the bold, dashed line in FIG. 9) enters the cartridge 300 through the air entry 341 and impinges against the central member 339 (or splitter), which causes the air to pass through both of the air apertures (337a, 337b). The central member 339 can be substantially centrally located above the air entry 341, and it can be referenced as a wedge in that it is effective to split the air stream into two separate streams as the air stream passes by the wedge. As discussed further below, vapor formed by the heater 320 mixes with the air coming through the air apertures (337a, 337b) to form an aerosol, which through one or more passages around the electrical contact 345a to enter the aerosol pathway 308 and proceed as already described above. The structure and placement of the single air entry 341 in the lower frame member 340 and the two air apertures (337a, 337b) in the bottom seal 336 can function together to provide a desired pressure drop through the cartridge 300 as air enters the cartridge, as the air mixes with formed vapor to create an aerosol, and as the aerosol passes through the aerosol pathway 308 and out the aerosol outlet 304.

Although not wishing to be bound by theory, it is believed that the splitting of the incoming air stream in combination with an aerosol pathway along one side of the cartridge can be effective to both “push” and “pull” formed vapor toward the aerosol pathway 308. The portion of the split air proximate to the aerosol pathway 308 can create a vacuum effect so that vapor in the vaporization chamber 360 is pulled toward the aerosol pathway, and the portion of the split air distal from the aerosol pathway can push vapor across the vaporization chamber. This creates dynamic mixing in the vaporization chamber for improved mixing of the vapor with the air to form an aerosol and for ensuring more complete removal of the vapor from the vaporization chamber so as to reduce condensation therein.

In some embodiments, additional structural elements can be utilized to further improving mixing of the incoming air with the formed vapor in the vaporization chamber 360. As seen in FIG. 7, the bottom seal 336 can include a barrier 334a that defines a side wall of the vaporization chamber 360. The barrier 334a is positioned on the side of the bottom seal 336 that is distal from the aerosol pathway 308 of the cartridge 300. The barrier 334a can be effective to reduce the effective volume of the vaporization chamber to a desired range so that mixing of air and vapor can occur simultaneously with the formed aerosol being drawn from the device through the aerosol outlet 304. The bottom seal 336 further can be arranged so that the bottom wall 336a defines a continuous pathway toward the aerosol pathway 308. In particular, the bottom wall 336a can define a lower surface profile relative to surrounding components of the bottom seal 336, as is further discussed below, and this lower surface profile can allow for a compact arrangement of the components of the cartridge 300 while maintaining a properly sized channel from the vaporization chamber 360 to the aerosol pathway 308. The side of the bottom seal 336 proximate to the aerosol pathway 308 thus can expressly exclude a barrier as is present on the opposing side of the bottom seal. In some embodiments, however, an island 334b can be present on the bottom wall 336a between the vaporization chamber 360 and the aerosol pathway 308, and the island can define two aerosol channels (334c, 334d). The island 334b can be effective to split the aerosol stream flowing from the vaporization chamber 360 toward the aerosol pathway 308, and such splitting can again be effective to achieve desired pressure drop through the device. As such, in some embodiments, the present disclosure can provide a device wherein an airflow pathway therethrough is split at least once or is split at least twice and, as such, an air stream passing through the airflow pathway is also split at least once or is split at least twice. The airflow pathway (and the air stream) may be split only in the direction that is substantially parallel to the longitudinal axis of the device, and such splitting may correspond to the splitting when air flows through the air entry 341 and around the central member 339 to flow separately through the two air apertures (337a, 337b) in the bottom seal 336. The airflow pathway (and the air stream) alternatively may be split only in a direction that is orthogonal to the longitudinal axis of the device, and preferably substantially perpendicular to the longitudinal axis of the device, and such splitting may correspond to the splitting when the mixture of air and vapor leaving the vaporization chamber 360 flows around the island 334b in the two channels (334c, 334d). In certain embodiments, the device can be configured so that the airflow pathway (and the air stream) may be split in both of the direction that is substantially parallel to the longitudinal axis of the device and a direction that is substantially orthogonal (and preferably substantially perpendicular) to the longitudinal axis of the device.

The split air arrangement described above relative to the air entry 341 and the two air apertures (337a, 337b) can be characterized as converting a single air stream into a plurality of air streams that are directed toward a surface of the heater 320 (or a surface of another embodiment of an atomizer) at a plurality of points that are laterally positioned relative to a geometric center of the surface of the heater (or other atomizer) and/or on two sides of a midline of the surface of the heater (or a surface of another embodiment of an atomizer). It is understood that the geometric center of the heater 320 can be the location that is approximately centered all of side-to-side, front-to-back, and diagonally across the surface of the heater/atomizer against which incoming air may impinge. Thus, the geometric center of the surface of the heater 320 can be at the overlapping point or area this is approximately centrally located on a line extending from the first end 322a to the second end 322b approximately midway between the front edge 323a and the back edge 323b of the heater and that is also approximately centrally located on a line extending from the front edge 323a to the back edge 323b approximately midway between the first end 322a and the second end 322b of the heater. It is likewise understood that a midline of the surface of the heater 320 may be a line that is approximately centrally located between the front edge 323a and the back edge 323b and extending side-to-side (i.e., from the first end 322a to the second end 322b) so that the surface of the heater is figuratively split into a front half and a rear half (defined as a long transverse midline) or may be a line that is approximately centrally located between the first end 322a and the second end 322b and extending front-to-back (i.e., from the front edge 323a to the back edge 323b) so that the surface of the heater is figuratively split into a left side and a right side (defined as a short transverse midline). The designations for a “long” or “short” transverse midline can relate to the dimensions of the heater 320 in that the heater can have a side-to-side length that is greater than a front-to-back length. In such arrangement, the side-to-side midline would be the long transverse midline, and the front-to-back midline would be the short transverse midline. Preferably, the air apertures (337a, 337b) are arranged so that the split airstreams approach a surface of the heater 320 at two locations that are not aligned with the geometric center of the heater. In some embodiments, the air apertures (337a, 337b) are arranged so that the split airstreams approach a surface of the heater 320 on alternate sides of the short transverse midline. Although less preferred, the air apertures (337a, 337b) may also be arranged so that the split airstreams approach a surface of the heater 320 on alternate sides of the long transverse midline. As noted above, however, any splitting elements that are utilized for splitting an air stream into a plurality of air streams can be positioned so that the plurality of air streams are directed toward a surface of an atomizer (e.g., the heater 320) at a plurality of points that are laterally positioned relative to the geometric center of the surface of the atomizer.

In some embodiments, one or more apertures extending through the bottom wall 336a of the bottom seal 336 can be configured to include one or more elements therearound that can be effective to reduce or eliminate leaking of liquid out of the cartridge 300. For example, as illustrated in FIG. 7, the air apertures (337a, 337b) in the bottom seal 336 have a raised profile relative to immediately adjacent portions of the bottom wall 336a of the bottom seal. As seen in the figure, the bottom wall 336a can define a floor across at least a portion of the top surface thereof, and this floor can be configured to trap condensed liquids. The raised profiles may thus form walls or baffles (335a, 335b) surrounding the respective air apertures (337a, 337b). The walls/baffles (335a, 335b) may be effective to substantially prevent liquid present on the floor defined by the bottom wall 336a from passing through the air apertures (337a, 337b) and leaking out of the cartridge 300. For example, vapor that is formed but does not fully exit the device may at least partially condense and may collect on the floor defined by the bottom wall 336a, and the walls/baffles (335a, 335b) can be effective to at least partially prevent the liquid from leaking through the air apertures (337a, 337b). While this configuration is illustrated in relation to the air apertures (337a, 337b), it is understood that the walls/baffles may be utilized for any aperture extending through the bottom wall 336a.

The heater 320 can be positioned between the bottom seal 336 and the upper frame member 332. The upper frame member 332 can sit substantially directly below the reservoir 306 and can include one or a plurality of slots 333 extending therethrough. The liquid transport element 310 can be disposed so as to at least partially extend through the upper frame member 332. In this manner, the liquid transport element 310 can transfer liquid from the reservoir 306 through the upper frame member 332 and to the heating member 320. The liquid transport element 310 can be formed of any suitable material for transport of the aerosol precursor liquid therethrough, such as by capillary action. For example, in some implementations the liquid transport element may be formed of fibrous materials (e.g., organic cotton, cellulose acetate, regenerated cellulose fabrics, glass fibers), porous ceramics, porous carbon, graphite, porous glass, sintered glass beads, sintered ceramic beads, capillary tubes, or the like. In other implementations, the liquid transport element 310 may be any material that contains an open pore network (i.e., a plurality of pores that are interconnected so that fluid may flow from one pore to another in a plurality of direction through the element). As further discussed herein, some implementations of the present disclosure may particularly relate to the use of non-fibrous transport elements. As such, fibrous transport elements may be expressly excluded. Alternatively, combinations of fibrous transport elements and non-fibrous transport elements may be utilized. Representative types of substrates, reservoirs or other components for supporting the aerosol precursor are described in U.S. Pat. No. 8,528,569 to Newton; U.S. Pat. App. Pub. Nos. 2014/0261487 to Chapman et al. and 2014/0059780 to Davis et al.; and U.S. Pat. App. Pub. No. 2015/0216232 to Bless et al.; which are incorporated herein by reference in their entireties. Additionally, various wicking materials, and the configuration and operation of those wicking materials within certain types of electronic cigarettes, are set forth in U.S. Pat. No. 8,910,640 to Sears et al.; which is incorporated herein by reference in its entirety. In some implementations, the liquid transport element may be formed partially or completely from a porous monolith, such as a porous ceramic, a porous glass, or the like. Example monolithic materials suitable for use according to embodiments of the present disclosure are described, for example, in U.S. Pat. No. 10,194,694 to Davis et al. and US Pat. No. 2014/0123989 to LaMothe, the disclosures of which are incorporated herein by reference in their entireties.

As shown in the figures, the heating member 320 can be configured to have a substantially flat profile (e.g., initially formed as a substantially planar element). Although in other implementations additional and/or differing contact features may be provided, the heater 320 of the depicted implementation includes a pair of contact holes 321a, 321b that are configured to connect the heater 320 to the electrical contacts (345a, 345b) of the cartridge 300. In the depicted implementation, the electrical contacts (345a, 345b) can be made of a conductive material and can be plated with nickel and/or gold. Examples of conductive materials include, but are not limited to, copper, aluminum, platinum, gold, silver, iron, steel, brass, bronze, graphite, conductive ceramic materials, and/or any combination thereof. In the depicted implementation, the contact holes 321a, 321b are configured to have an inner diameter that is less than an outer diameter of the mating portions of the electrical contacts (345a, 345b). In some implementations, the contact holes may include one or more features (e.g., one or more fingers or extensions) that create an effective inner diameter that is less than an outer diameter of the mating portion of the electrical contacts (345a, 345b). In such a manner, the contact holes 321a, 321b of the heater 320 may create an interference fit with the upper ends of the electrical contacts (345a, 345b) such that the heater 320 may maintain electrical contact with the electrical contacts (345a, 345b). Alternative contact holes are depicted in the perimeter member 322, and it is understood that the contact holes described above may reference the holes included in the perimeter member.

Although other implementations may differ, in the depicted implementation the heating member 320 includes a first end 322a, a second end 322b, and a heater loop 323 connecting the first end and the second end, the heater loop defining a front edge 323a and a back edge 323b. It is understood that the labels “first”, “second”, “front”, and “back” are used for clarity, are not intended to limit orientation of the heater 320 in the device, and the ends and edges may be flipped as desired. In the depicted implementation, the heater 320 has a long axis extending between the first end 322a and the second end 322b that can align with the long transverse midline discussed above, and the heater has a short axis extending between the first edge 323a and the second edge 323b that can align with the short transverse midline discussed above.

The heater loop of the depicted implementation comprises a serpentine pattern of heater traces that are connected at respective ends thereof and that extend substantially transverse to a longitudinal axis of the heating member to connect the first end to the second end. While in some implementations the heater traces may be solid, the heater traces of the depicted implementation comprise a plurality of split traces so that the splits define sections of the heater with smaller dimensions (e.g., width and thickness) relative to the overall size of the heater traces. The split traces can be useful to increase heating surface area while also increasing useful volume for generation of vapor between the trace sections. In the depicted implementation, the edges of the heating member are substantially solid and the plurality of split traces are located in a central area of the heating member. In such a manner, the heater loop of the depicted implementation may be configured to concentrate heat in an area of the heating element configured to be in contact with the liquid transport element 310. In the depicted implementation, the liquid transport element 310 and the heating member 320 can define an atomizing assembly. The upper frame member 332 and the bottom seal 336 can together define a vaporization chamber 360 (see FIG. 9).

It should be noted that some implementations need not include a heater and/or a liquid transport element and thus may be configured to generate an aerosol in an alternative manner Some examples of atomization assemblies that generate aerosols in alternative manners can be found, for example, in U.S. application Ser. No. 16/544,326, filed on Aug. 19, 2019, and titled Detachable Atomization Assembly for Aerosol Delivery Device, which is incorporated herein by reference in its entirety.

In the depicted implementation, the heating member 320 may be made of a metal material, such as a stainless steel material, including, but not limited to, 316L, 316, 304, or 304L stainless steel. In other implementations, the heating member may be made of a different material, such as, for example, Kanthal (FeCrAl), Nichrome, Molybdenum disilicide (MoSi₂), molybdenum silicide (MoSi), Molybdenum disilicide doped with Aluminum (Mo(Si,Al)₂), titanium, platinum, silver, palladium, alloys of silver and palladium, graphite and graphite-based materials (e.g., carbon-based foams and yarns). In further implementations, the heating member may be formed from conductive inks, boron doped silica, and/or ceramics (e.g., positive or negative temperature coefficient ceramics). Other types of heaters may also be utilized, such as laser diodes or microheaters. A laser diode can be configured to deliver electromagnetic radiation at a specific wavelength or band of wavelengths that can be tuned for vaporization of the aerosol precursor composition and/or tuned for heating a liquid transport element via which the aerosol precursor composition may be provided for vaporization. The laser diode can particularly be positioned so as to deliver the electromagnetic radiation within a chamber, and the chamber may be configured to be radiation-trapping (e.g., a black body or a white body). Suitable microheaters are described in U.S. Pat. No. 8,881,737 to Collett et al., which is incorporated herein by reference in its entirety. Microheaters, for example, can comprise a substrate (e.g., quartz, silica) with a heater trace thereon (e.g., a resistive element such as Ag, Pd, Ti, Pt, Pt/Ti, boron-doped silicon, or other metals or metal alloys), which may be printed or otherwise applied to the substrate. A passivating layer (e.g., aluminum oxide or silica) may be provided over the heater trace. Other heaters are described in U.S. Pat. App. Pub. No. 2016/0345633 to DePiano et al., which is incorporated herein by reference in its entirety.

The bottom cap 350 of the depicted implementation is configured to be secured to the distal end of the tank body 302 via snap features included on one or both of the bottom cap 326 and tank body, although other attachment methods are possible (e.g., via adhesives, heat staking/welding, ultrasonic welding, etc.). In the depicted implementation, the bottom cap 350 of the cartridge 300 includes a central cut-out 351 through which one or more of the air entry 341 and the electrical contacts (345a, 345b) may be accessible.

When the cartridge 300 of the depicted implementation is coupled with the control device 200, the electrical connection between the control device 200 and the heater 320 of the cartridge 300 (via the conductive pins 236A, 236B of the control device 200 and the electrical connectors (345a, 345b) of the cartridge) allows the control body 200 to direct electrical current to the heater 320. In the depicted implementation, this may occur when a puff on the aerosol delivery device 100 is detected (or, in other implementations, via actuation by the user, such as, for example, via a pushbutton). When a user of the aerosol device 100 of the depicted implementation draws on the cartridge 300, inlet airflow is directed into the device 100 via a gap between the cartridge 300 (e.g., an outer wall of the cartridge 300) and the control device 200 (e.g., an inner wall of the control device 200 defining the receiving chamber 230 thereof).

As a user draws on the device 100, the air that enters the gap between the cartridge 300 and the control device 200 travels downward around the outside of the cartridge 300 and below the bottom cap 350 thereof. The air that enters through the air entry 341 then can proceed through the cartridge as already described above. When a draw is detected by the pressure sensor 240, the control component 214 directs current through the heater 320 in order to heat the heater.

Although in some implementations a cartridge and a control device may be provided together as a complete aerosol delivery device generally, these components may be provided separately. For example, the present disclosure also encompasses a disposable unit for use with a reusable unit. In specific implementations, such a disposable unit (which may be a cartridge as illustrated in the appended figures) can be configured to engage a reusable unit (which may be a control device as illustrated in the appended figures). In still other configurations, a cartridge may comprise a reusable unit and a control device may comprise a disposable unit.

Although some figures described herein illustrate a cartridge and a control device in a working relationship, it is understood that the cartridge and the control device may exist as individual components. Accordingly, any discussion otherwise provided herein in relation to the components in combination also should be understood as applying to the control device and the cartridge as individual and separate components.

In another aspect, the present disclosure may be directed to kits that provide a variety of components as described herein. For example, a kit may comprise a control device with one or more cartridges. A kit may further comprise a control device with one or more charging components. A kit may further comprise a control device with one or more batteries. A kit may further comprise a control device with one or more cartridges and one or more charging components and/or one or more batteries. In further implementations, a kit may comprise a plurality of cartridges. A kit may further comprise a plurality of cartridges and one or more batteries and/or one or more charging components. In the above implementations, the cartridges or the control devices may be provided with a heating member inclusive thereto. The inventive kits may further include a case (or other packaging, carrying, or storage component) that accommodates one or more of the further kit components. The case could be a reusable hard or soft container. Further, the case could be simply a box or other packaging structure.

Use herein of the terms “about”, “approximately”, and “substantially” are intended to indicate that a parameter is exactly as recited or varies from the exactly recited condition by relatively small deviations that would be recognized as arising from typical manufacturing methods and/or sampling errors. For example, a value stated as being “about” or “approximately” a stated value is intended to encompass the exactly stated value as well as slight deviations therefrom, such as +/−3%, +/−2%, +/−1%, +/−0.5%, or +/−0.1% of the exactly stated value. Likewise, an item that is discussed herein as having “substantially” a stated condition is intended to encompass the exactly stated condition as well as slight deviations therefrom that may arise from manufacturing methods or the like.

Many modifications and other embodiments of the disclosure will come to mind to one skilled in the art to which this disclosure pertains having the benefit of the teachings presented in the foregoing descriptions and the associated drawings. Therefore, it is to be understood that the disclosure is not to be limited to the specific embodiments disclosed herein and that modifications and other embodiments are intended to be included within the scope of the appended claims. Although specific terms are employed herein, they are used in a generic and descriptive sense only and not for purposes of limitation.

1. A cartridge for use in an aerosol delivery device, the cartridge comprising:
a body defining a reservoir and defining an aerosol pathway in fluid communication with an aerosol outlet;
an air entry;
an atomizer;
an aerosol forming chamber; and
an air pathway extending from the air entry, through the aerosol forming chamber, the aerosol pathway, and the aerosol outlet;
wherein the cartridge includes one or more splitting elements positioned so as to be effective to split an air stream passing through the air pathway prior to entering the aerosol forming chamber.

2. (canceled)

3. The cartridge of claim 1, wherein the one or more splitting elements is positioned so that the air stream is split into a plurality of air streams that are directed toward a surface of the atomizer at a plurality of points that are laterally positioned relative to a geometric center of the surface of the atomizer.

4. The cartridge of claim 1, wherein the atomizer includes a surface with a midline that extends from a front edge of the atomizer to a back edge of the atomizer and that is approximately centrally located between a first end of the atomizer and a second end of the atomizer, and wherein the one or more splitting elements is positioned so that the air stream is split into two air streams that are directed toward the surface of the atomizer on each of two sides of the midline.

5. The cartridge of claim 1, wherein the one or more splitting elements comprises a wedge that is substantially centrally located above the air entry.

6. The cartridge of claim 1, wherein the cartridge comprises an upper frame member positioned proximate the reservoir, a lower frame member engaging an opening at an end of the body opposite from the aerosol outlet, and a bottom seal positioned in the body between the upper frame member and the lower frame member.

7. The cartridge of claim 6, wherein the air entry is defined in the lower frame member.

8. The cartridge of claim 7, wherein the bottom seal includes two air apertures.

9. The cartridge of claim 8, wherein one or both of the following conditions are met:
the one or more splitting elements comprises a central member positioned in the bottom seal between the two air apertures;
the two air apertures are substantially symmetrically arranged relative to the air entry.

10. (canceled)

11. The cartridge of claim 6, wherein the aerosol forming chamber is defined between the upper frame member and the bottom seal.

12. The cartridge of claim 11, wherein the bottom seal comprises an island positioned between the aerosol forming chamber and the aerosol pathway of the body.

13. The cartridge of claim 12, wherein the island is effective to split an aerosol stream from the aerosol forming chamber into two streams prior to passage of the aerosol stream into the aerosol pathway.

14. The cartridge of claim 6, wherein the bottom seal comprises a bottom wall that defines a floor across at least a portion of a top surface of the bottom seal.

15. The cartridge of claim 14, wherein the floor of the bottom seal comprises one or more components effective to trap liquid therein.

16. The cartridge of claim 15, wherein the one or more components effective to trap liquid comprise one or more baffles that individually completely surround one or more openings though the bottom seal.

17. The cartridge of claim 16, comprising baffles that individually completely surround a plurality of air apertures through the bottom seal.

18. The cartridge of claim 6, wherein the bottom seal comprises an outer edge that is in contact with an inner surface of an outer wall of the tank body.

19. The cartridge of claim 18, wherein the outer edge of the bottom seal comprises a plurality of ribs.

20. The cartridge of claim 1, wherein the cartridge includes as a first splitting element, the splitting element that is positioned so as to be effective to split the air stream passing the through the air pathway prior to entering the aerosol forming chamber, and wherein the cartridge further includes a second, separate splitting element effective to split the air stream a second time while passing through the air pathway.

21. The cartridge of claim 20, wherein the first splitting element and the second splitting element are positioned in the cartridge so that the air stream is split in a direction that is substantially parallel to a longitudinal axis of the cartridge and is split in a direction that is substantially orthogonal to the longitudinal axis of the cartridge.

22. The cartridge of claim 1, wherein the atomizer comprises one or both of a heater and a liquid transport element.

23. (canceled)

24. A cartridge for use in an aerosol delivery device, the cartridge comprising:
a body including an outer wall and defining a reservoir, an aerosol passage in fluid communication with an aerosol outlet, and an opening at a bottom end thereof, the opening being defined by the outer wall; and
a sealing member positioned within the tank body between the reservoir and the opening at the bottom of the tank body, the sealing member having an outer edge that is in contact with an inner surface of the outer wall of the tank body, wherein the sealing member includes a bottom wall with at least one aperture therethrough, the at least one aperture being surrounded by a baffle that extends upward from the bottom wall.
